(19) United States
(12) Patent Application Publication
(10) Pub. No.: US 2026/0096811 A1
(43) Pub. Date: Apr. 9, 2026

(54) VARIABLE-SHAPE LAPAROSCOPIC INSTRUMENT

(71) Applicant: NUtech Ventures, Lincoln, NE (US)

(72) Inventors: Carl Nelson, Lincoln, NE (US); Lily Scholnik, Silver Spring, MD (US)

(21) Appl. No.: 19/287,519

(22) Filed: Jul. 31, 2025

Related U.S. Application Data
(60) Provisional application No. 63/678,982, filed on Aug. 2, 2024.

(51) Int. Cl. A61B 17/00

(52) U.S. Cl. CPC A61B 17/00234; A61B 2017/00323; A61B 2017/00464

(57) ABSTRACT

A surgical instrument may include a tool handle that includes a tool control, and a tool shaft. The tool shaft may include a proximal end mechanically coupled to the tool handle, a distal end disposed opposite to the proximal end; and a plurality of shaft segments. The surgical instrument may include a tool disposed on the distal end of the tool shaft and mechanically coupled to the tool control. The surgical instrument may include a plurality of joints configured to couple at least two shaft segments of the plurality of shaft segments, one or more joints of the plurality of joints including a male connector and a female connector configured to couple to a mating interface, wherein a rotation of the male connector relative to the female connector causes an articulation of the tool relative to the tool handle.

### CROSS-REFERENCE TO RELATED APPLICATION

The present application claims the benefit under 35 U.S.C. § 119(e) of U.S. Provisional Application Ser. No. 63/678,982, filed Aug. 2, 2024, which is incorporated herein by reference in the entirety.

### GOVERNMENT SUPPORT

This invention was made with government support under EEC2050587 awarded by the National Science Foundation. The government has certain rights in the invention.

### TECHNICAL FIELD

The present invention generally relates to surgical instruments and, more particularly, to laparoscopic surgical instruments.

### BACKGROUND

Laparoscopic surgery is an improvement over traditional open surgery as it improves patient recovery time and post-operative pain levels. Laparoscopy traditionally involves multiple entry ports, each with its own rigid trocar (e.g., insertion port), with a single tool per port. An emerging frontier of laparoscopy is single-incision laparoscopic surgery (SILS), in which tools pass through a single, larger port made through the navel. This procedure most notably results in a scarless outcome, but also has the potential to further improve post-surgical outcomes for patients in terms of pain levels and recovery time, as well as infection risk.

The potential benefits of SILS are still being explored, but are currently limited in part by the technological lag of laparoscopic tools. Triangulation, in which multiple tools and an endoscope converge on the surgical site to allow easy visualization and an open workspace, is a key principle of laparoscopic procedures, which is lost in the SILS method. A significant limitation of laparoscopy is that, due to the shape of the tools, a straight tool-path from the surgical site is required. In the SILS method, this creates several issues. Tool handle crowding outside of the patient creates added technical complexity for the surgeon. Inside the patient, the single port causes a loss of triangulation that also raises the learning curve for the surgeon.

Laparoscopic procedures performed using the single-port method take longer than their traditional laparoscopic counterparts. There is also reduced tool mobility in the port, and a greater incidence of tool crossing. The sum of these issues is that, in general, only experienced surgeons can perform the single-port method, and on a limited subset of patients. The benefits of the single-port method may become clearer as more surgeons become experienced in the method, and as laparoscopic tool design addresses its current limitations.

Therefore, it would be advantageous to provide a system and method that overcomes the shortcomings described above.

### SUMMARY

In embodiments, a surgical instrument is disclosed. In one or more embodiments, the surgical instrument includes a tool handle including a tool control. In one or more embodiments, the surgical instrument includes a tool shaft coupled at a proximal end to the tool handle. In one or more embodiments, the tool shaft includes a plurality of shaft segments, wherein the plurality of shaft segments includes a lumen extending along a length of the plurality of shaft segments. In one or more embodiments, the tool shaft includes a plurality of joints configured to couple adjacent shaft segments of the plurality of shaft segments. In one or more embodiments, one or more joints of the plurality of joints includes a male connector including a male mating surface and a female connector including a female mating surface and configured to couple to the male connector at a mating interface, wherein a rotation of the male connector relative to the female connector causes an articulation of a tool relative to the tool handle. In one or more embodiments, the surgical instrument includes a tool disposed on a distal end of the tool shaft and mechanically coupled to the tool control, wherein actuation of the tool control causes the tool to perform a function. In one or more embodiments, the surgical instrument includes a lock assembly disposed on the tool handle and configured to lock the tool shaft, wherein locking the tool shaft prevents the male connector from rotating relative to the female connector.

In embodiments, a method for configuring a surgical instrument in preparation for a laparoscopic surgery is disclosed. In one or more embodiments, the method includes unlocking a lock assembly of a tool shaft of the surgical instrument, the tool shaft including at least one joint, wherein the at least one joint includes a female connector and a male connector, wherein unlocking the lock assembly causes the female connector to be rotatable relative to the male connector. In one or more embodiments, the method includes rotating the female connector relative to the male connector, wherein rotating the female connector relative to the male connector causes the two shaft segments to reconfigure into a surgical configuration. In one or more embodiments, the method includes locking the lock assembly into the surgical configuration, wherein locking the lock assembly into the surgical configuration prevents the female connector from rotating relative to the male connector. In one or more embodiments, the method includes inserting the tool shaft into a surgical port.

### BRIEF DESCRIPTION OF THE DRAWINGS

The numerous advantages of the disclosure may be better understood by those skilled in the art by reference to the accompanying figures.

FIG. 1A illustrates a side view of a surgical instrument, in accordance with one or more embodiments of the present disclosure.

FIG. 1B illustrates a cutaway side view of a handle of the surgical instrument and a proximal shaft segment, in accordance with one or more embodiments of the present disclosure.

FIG. 2A illustrates a side view of a tool shaft and tool, in accordance with one or more embodiments of the disclosure.

FIG. 2B illustrates three configurations of the tool shaft 104a-c, in accordance with one or more embodiments of the disclosure.

FIGS. 3A-3B illustrate perspective views of an assembled straight joint and an unassembled straight joint, respectively, in accordance with one or more embodiments of the disclosure.

FIGS. 4A-4B illustrate perspective views of an assembled angulated joint and an unassembled angulated joint, respectively, in accordance with one or more embodiments of the disclosure.

FIG. 5 illustrates semitransparent side-views of joints, in accordance with one or more embodiments of the disclosure.

FIG. 6A illustrates a joint that includes an expansion lock mechanism where the female and male connectors are unlocked through compression, in accordance with one or more embodiments of the disclosure.

FIG. 6B illustrates a joint that includes a compression lock mechanism wherein the female and male connectors are unlocked through releasing compression, in accordance with one or more embodiments of the disclosure.

FIG. 7 illustrates a process flow diagram depicting a method for configuring a surgical instrument in preparation for a laparoscopic surgery, in accordance with one or more embodiments of the disclosure.

FIGS. 8A-8C illustrates finite element analysis (FEA) results for tested joints, in accordance with one or more embodiments of the disclosure.

FIG. 9 illustrates a graph depicting the possible positional configurations of the surgical tool shown in FIG. 1A, in accordance with one or more embodiments of the disclosure.

### DETAILED DESCRIPTION

Reference will now be made in detail to the subject matter disclosed, which is illustrated in the accompanying drawings. The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

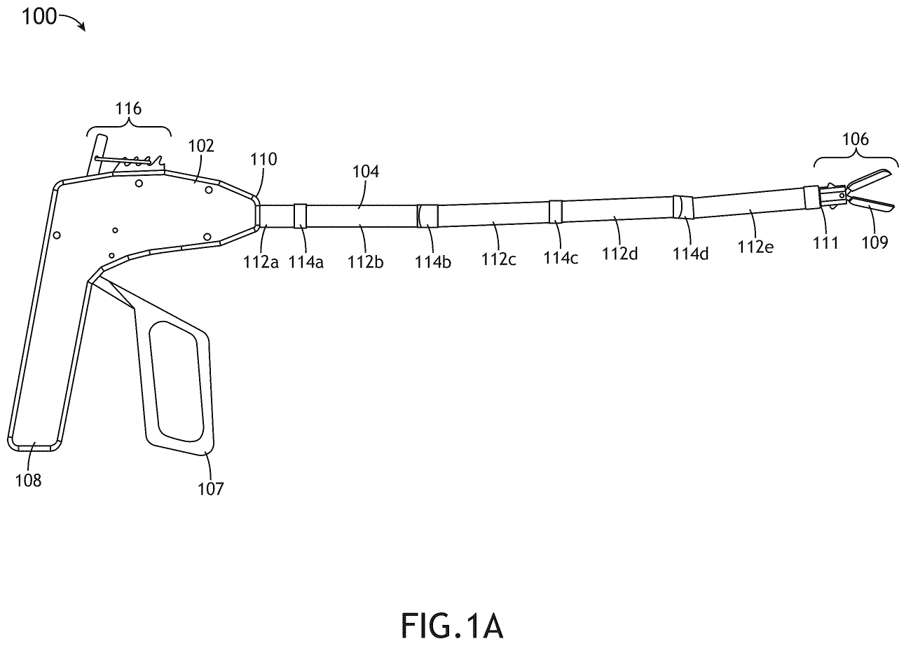

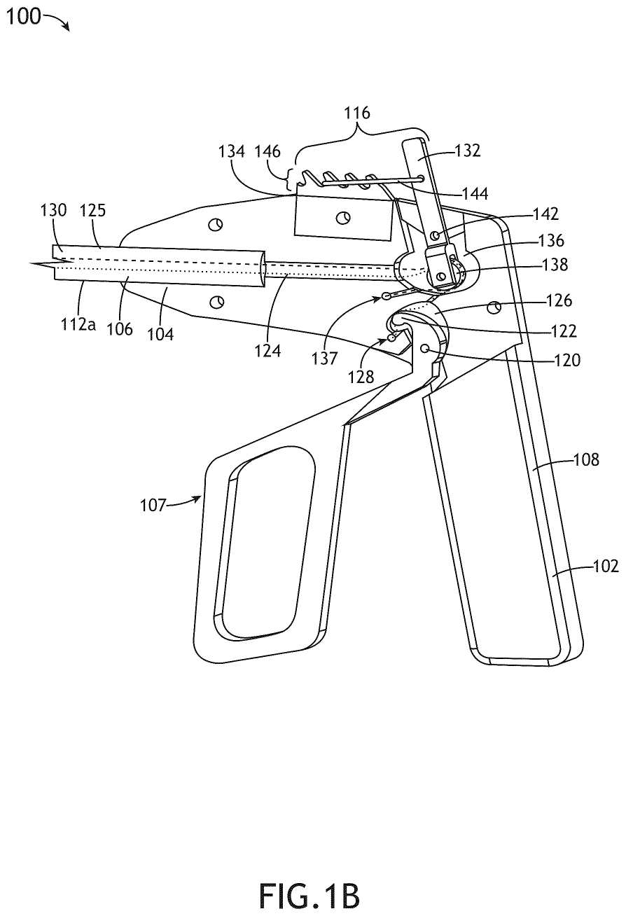

FIGS. 1A-9 generally illustrate a laparoscopic instrument, in accordance with one or more embodiments of the present disclosure.

Embodiments of the present disclosure are directed to a surgical instrument in laparoscopic surgeries. The surgical instrument may include a tool handle and tool shaft that controls a tool, such as a grasping tool, for insertion into a laparoscopic port. The tool shaft may include a set of rotating and articulating joints that can be manually adjusted, causing the tool to articulate relative to the tool handle. The configurable tool shaft allows a surgeon to configure the surgical instrument into one of a large number of possible settings that best fits the needs of the surgeon during a specific surgery.

FIG. 1A illustrates a side view of a surgical instrument 100, in accordance with one or more embodiments of the present disclosure. The surgical instrument 100 may be used in laparoscopic surgeries, with a portion of the surgical instrument 100 being insertable into a surgical insertion port. The surgical instrument 100 may include a handle 102 and a tool shaft 104 that supports and controls a tool 106, such as a grasping tool (e.g., a grasper). The surgical instrument 100 may include other types of tools 106, including, but not limited to, trocars, laparoscopes, scissors, clamps, staplers, needle holders, and electrosurgical devices. The surgical instrument may include a tool control (e.g., a trigger 107) for operating the tool 106. For example, actuation of the tool control may cause the tool to perform a function, such as grasping, stapling, or cutting. For instance, by clenching the trigger 107 against a grip 108 of the handle 102, a cable operationally coupled to one or more jaws 109 of the grasper may be pulled, causing the jaws 109 to close. Other types of tool controls include, but are not limited to, switches and push buttons. The tool shaft 104 may be coupled to the handle 102 at a proximal end 110, and to the tool 106 at a distal end 111.

In embodiments, the tool shaft 104 includes a plurality of shaft segments 112a-e coupled to each other via a plurality of joints 114a-d that coupled adjacent shaft segments 112 within the tool shaft 104 (e.g., the tool shaft 104 may include at least two shaft segments 112 coupled by a joint 114). One or more shaft segments 112 of the plurality of shaft segments 112a-e may include a lumen that extends along a length of the one or more shaft segments.

In embodiments, one or more joints 114a-d may be configured to rotate and/or articulate, enabling two adjacent shaft segments 112 coupled by one of the one or more joints 114 to rotate independently of each other. The tool 106 may be coupled to the shaft segment 112e (e.g., a distal shaft segment) via a joint 114 or a direct connection. Similarly, the handle 102 may be coupled to the shaft segment 112a (e.g., a proximal shaft segment) via a joint 114 or a direct connection. The plurality of joints 114a-d allows the shaft segments 112a-e to be rotatable relative to each other, resulting in a large number of possible shaft configurations. Once a shaft configuration is finalized for a surgical procedure, the shaft segments 112a-e may be locked into place via a lock assembly 116.

An adjustment of one or more joints 114a-d may enable the proximal shaft segment 112a and the distal shaft segment 112e to be angularly displaced relative to each other (e.g., via their respective longitudinal axes). For example, the proximal shaft and the distal shaft may be configured to be angularly displaced in a range of zero degrees to 120 degrees, angularly displaced in a range of zero degrees to 90 degrees, or angularly displaced in a range of zero degrees to 60 degrees, or approximate values.

In embodiments, the surgical instrument 100 includes transmission elements (e.g., gears and motors) that drive the set of joints 114, such as during surgery. In embodiments, the surgical instrument 100 includes static elements that allow a one-time preoperative manual joint manipulation.

The surgical instrument 100 may include any number of shaft segments 112. For example, the surgical instrument 100 may include two or more shaft segments 112, three or more shaft segments 112, four or more shaft segments 112, five or more shaft segments 112, or six or more shaft segments 112. For example, the surgical instrument 100 may include five shaft segments 112a-e.

FIG. 1B illustrates a cutaway side view of the handle 102 of the surgical instrument 100 and the proximal shaft segment 112a, in accordance with one or more embodiments of the present disclosure. The trigger 107 may be coupled to the handle 102 via a pivot joint 120. The trigger 107 may also include a trigger cam 122 that is in contact with a cable 124 (e.g., a control cable, depicted as a dotted line), the cable 124 may be operatively attached to the tool 106 via a lumen 125 of the tool shaft 104, with both the trigger cam 122 and a portion of the cable 124 disposed within a first cable anchoring channel 126 (e.g., with an end of the cable 124 secured at a first anchor point 128). Upon pulling the trigger 107 toward the grip 108, the cable 124 is pulled by the cam 122, activating the tool 106. For example, pulling the cable 124 may cause the jaws 109 to close.

In embodiments, the plurality of shaft segments 112a-e of the tool shaft 104 are locked into a lock position (e.g., for a surgical configuration) via a cable 130 (e.g., a joint cable, depicted as a dashed line) coupled to the lock assembly 116, the lock assembly 116 further including a pulley lever 132 and ratchet lock 134. For example, the cable 130 may be coupled via a second cable anchoring channel 136 at a second anchor point 137 and wrapped around, or otherwise coupled to, one of one or more pulley wheels 138 of the pulley lever 132 and anchored to a second cable anchoring channel 136. Upon pulling of the pulley lever 132, the pulley lever 132 may pivots along a lever pivot joint 142, causing the pulley wheel 138 to move away from the distal end 111 of the tool shaft 104. The movement of the pulley wheel 138 may cause the cable 130 to tighten, securing the shaft segments 112a-e (e.g., the single cable 130 pulls all shaft segments 112 together, locking them). The tightened cable 130 may be secured into the lock position by a clip 144 that couples the pulley lever 132 to one of several ratchet positions 146 of the ratchet lock 134. One or more of the pulley wheels 138 may further be operatively coupled to the cable 124, allowing the lever 132 to adjust the tension of both cables 124, 130 simultaneously.

The ratcheted lock 134 may have multiple ratchet positions 146 that enable cable adjustment and/or adjustment of shaft joints 114. For example, changes in the articulation of the joints 114 may change the length of the cable path, requiring tightening of the cables 124, 130 via a different ratchet position 146 that was used before articulation of the joints 114. In another example, the ratcheted lock 134 may have a moderately secure ratcheted position 146 that loosens the tool shaft 104 enough so that a joint 114 can be adjusted, and a highly secure ratcheted position that prevents the joints 114 from adjusting during surgery.

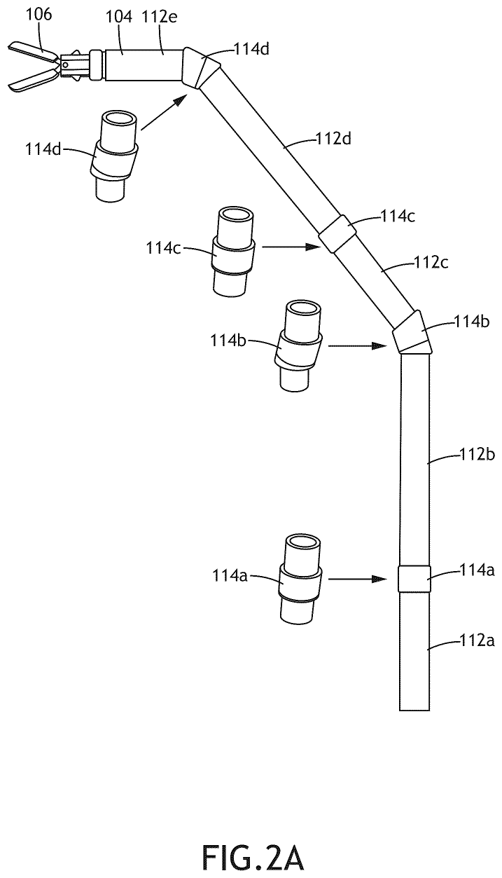

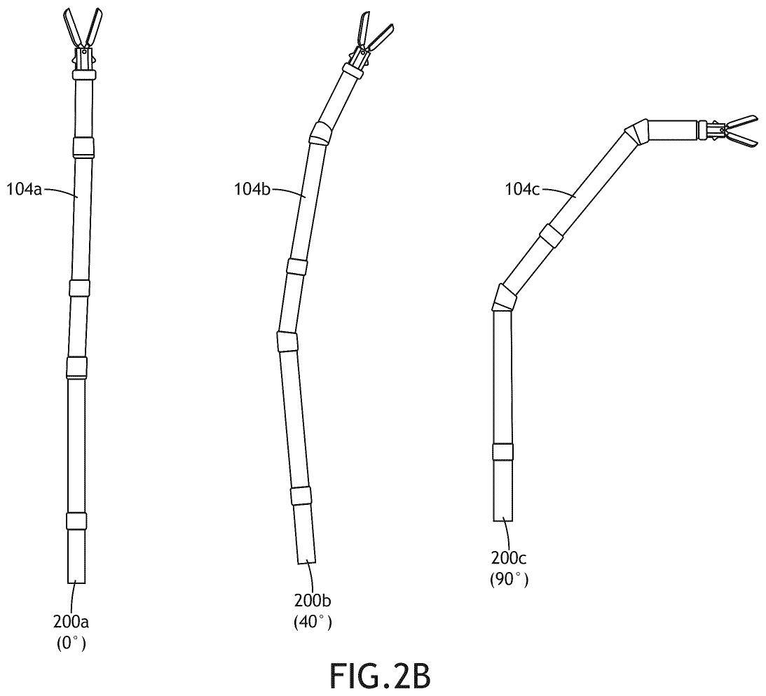

FIG. 2A illustrates a side view of a tool shaft 104 and tool 106, in accordance with one or more embodiments of the disclosure. Arrows illustrate the type of joints 114a-d that are integrated into the tool shaft 104. The joints 114a-d may include one or more straight joints 114a, 114c that allow only rotation between two adjacent shaft segments 112, one or more angulated joints 114b, 114d that cause both a rotation and angular displacement between two shaft segments 112, or a mixture of both. For example, the tool shaft 104 may include alternating straight joints 114a, 114c, and angulated joints 114b, 114d, as shown in FIG. 2A.

FIG. 2B illustrates three configurations 200a-c of the tool shaft 104a-c, in accordance with one or more embodiments of the disclosure. For example, by selectively rotating the joints 114, the tool shaft 104 can switch from a straight configuration 200a, having no bend, to a slightly bent configuration 204b with an approximately 40° bend, or to a right-angle configuration 204c having a 90° bend.

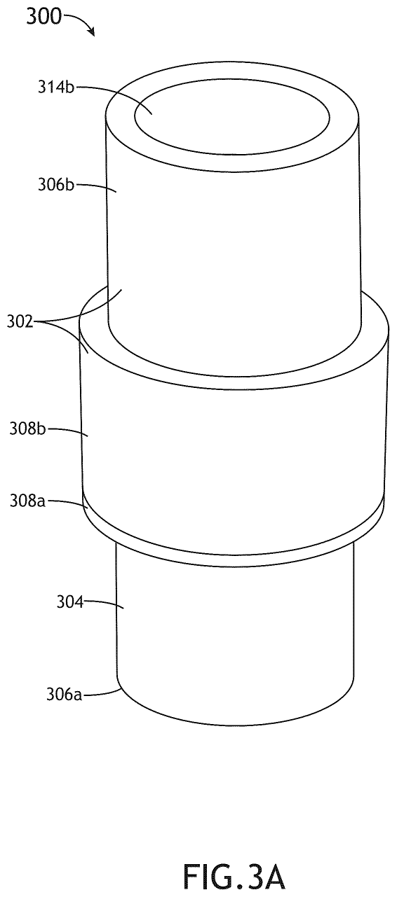

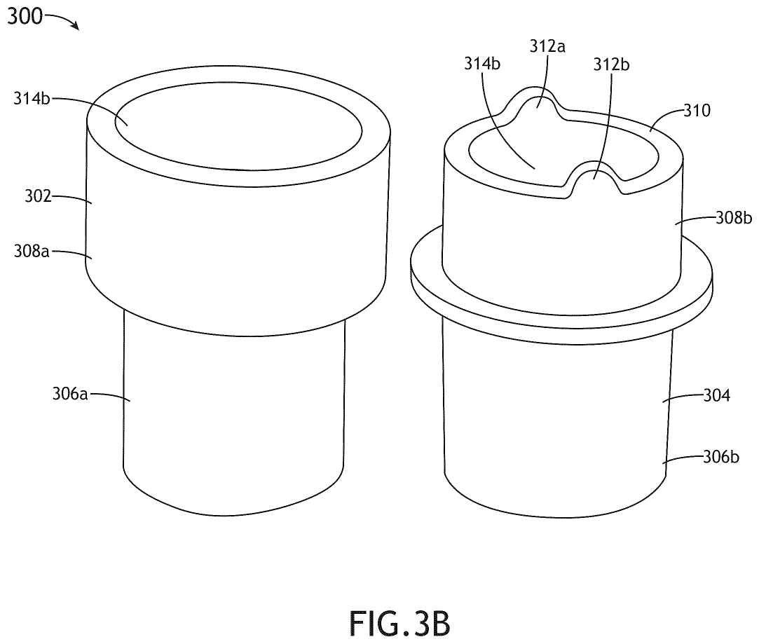

FIGS. 3A and 3B illustrate perspective views of an assembled straight joint 300 and an unassembled straight joint 300, respectively, in accordance with one or more embodiments of the disclosure. In embodiments, the straight joint 300 includes a straight female connector 302 and a straight male connector 304 (e.g., the straight male connector 304 being inserted into the female connector 302 in FIG. 3B. The straight female connector 302 and the straight male connector 304 may include respective support sections 306a-b that are insertable into the ends of the shaft segments 112 (e.g., the support sections 306a-b fit within the lumen 125 of the tool shaft 104).

The straight female connector 302 and the straight male connector 304 may include respective base sections 308a-b that support respective mating interfaces that engage with each other. For example, the straight male connector 304 may include a male mating surface 310 that includes one or more raised portions 312a-b that interact and lock with the mating interface of the straight female connector 302 (not shown in FIGS. 3A-3B). Both the straight female connector 302 and the straight male connector 304 may include lumens 314a-b that permit the stringing of cables 124, 130 through the joint 300.

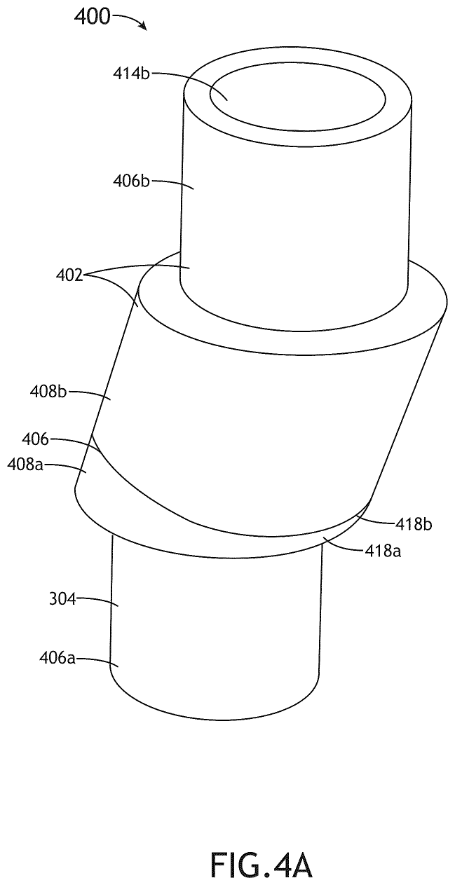

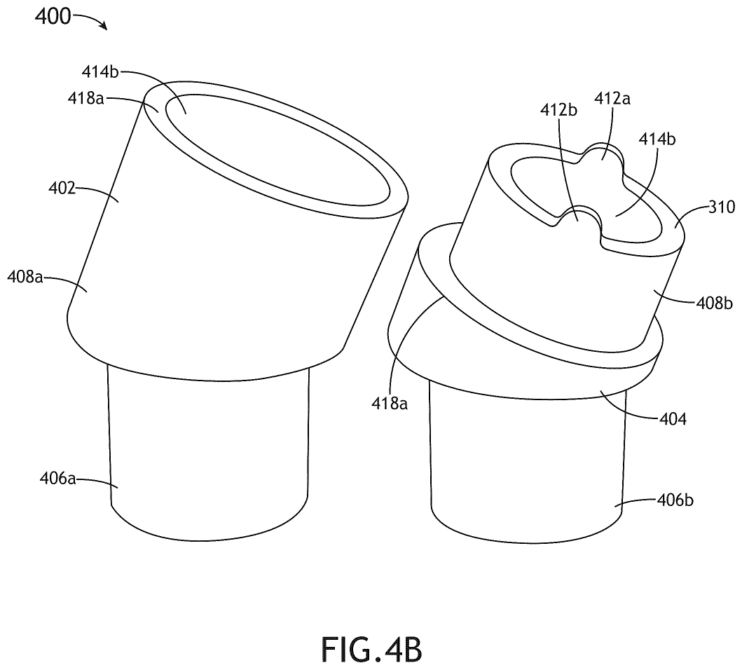

FIGS. 4A and 4B illustrate perspective views of an assembled angulated joint 400 and an unassembled angulated joint 400, respectively, in accordance with one or more embodiments of the disclosure. In embodiments, the angulated joint 400 includes an angulated female connector 402 and an angulated male connector 404 (e.g., the angulated male connector 404 being inserted into the angulated female connector 402 in FIG. 4B. The angulated female connector 402 and the angulated male connector 304 may include respective support sections 406a-b that are insertable into the ends of the shaft segments 112 (e.g., the support sections 406a-b fit within the lumen 125 of the tool shaft 104.

The angulated female connector 402 and the angulated male connector 404 may include respective base sections 408a-b that support respective mating interfaces. For example, the angulated male connector 404 may include a male mating surface 410 that includes one or more raised portions 412a-b that interact and lock with the mating interface of the angulated female connector 402 (not shown in FIGS. 4A-4B). Both the angulated female connector 402 and the angulated male connector 404 may include lumens 414a-b that permit the stringing of cables 124, 130 through the joint 400.

In embodiments, when the tool shaft 104 is in an unlocked configuration the female connectors 302, 402 and the male connectors 304, 404, of one or more joints 114 are configured to be rotate relative to one another about a shared axis of rotation. For example, when unlocked, the shaft segments 112 are joints 114 are loosely held together by the cable 130, permitting the one or more joints 114 to be adjusted without being removed from the tool shaft 104. When the tool shaft is in a locked configuration, the female connectors 302, 402 cannot rotate relative to the male connectors 304, 404.

In embodiments, the female connectors 302, 402 and the male connectors 304, 404 include an outer locking interface 416 that further supports mating and control of slippage between the female connectors 302, 402 and the male connectors 304, 404, as shown in FIG. 4A-4B. For example, the female connectors 302, 402 and the male connectors 304, 404 may include respective interlocking surfaces 418a-b.

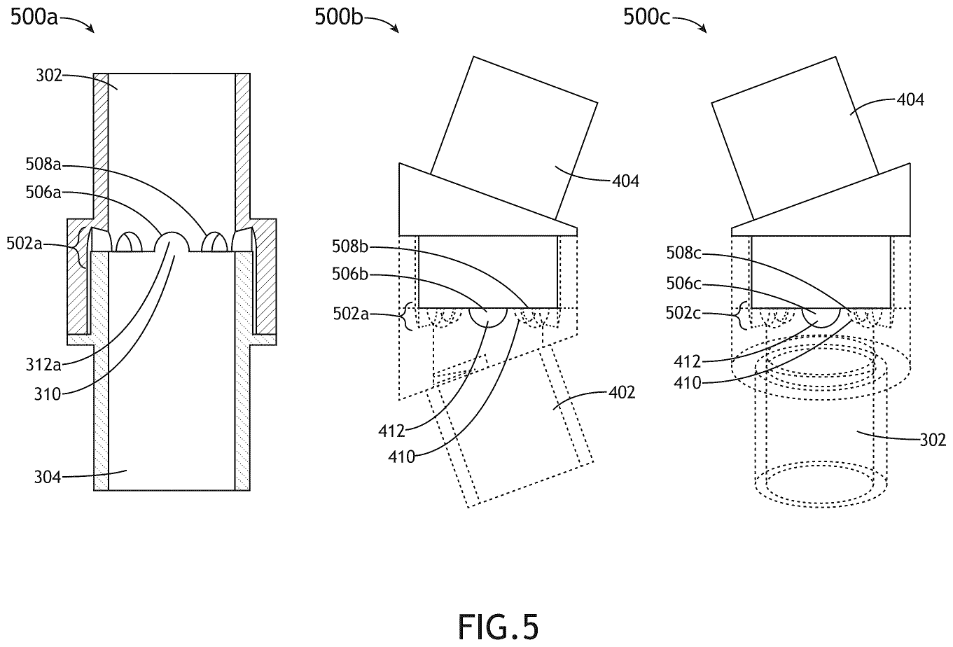

FIG. 5 illustrates semitransparent side-views of joints 500a-c, in accordance with one or more embodiments of the disclosure. For example, joint 500a may include a straight female connector 302 and a straight male connector 304. In another example, joint 500b includes an angulated female connector 402 and an angulated male connector 404. In embodiments, joint 500c includes both a straight component and an angulated component (e.g., a hybrid joint 500c). For example, joint 500c may be configured as a hybrid joint that includes a straight female connector 302 and an angulated male connector 404. A joint 500 that includes an angulated female connector 402 and a straight male connector 304 is also possible.

Referring to FIG. 5, joints 500a-c may include mating interfaces that secure the relative positions of the respective connectors. For example, one or more raised portions 312a, 412a of the male mating surface 310, 410 may fit within one or more notches 506a-c of a female mating surface 508a-c. Other types of mating interfaces (e.g., raised portions and notches) are possible. Therefore, the description herein should not be interpreted as a limitation of the present disclosure, but merely an illustration. The mating interfaces may utilize any number of notches 506 and raised portions 312. For example, the mating interfaces may include two or more notches 506, four or more notches 506, six or more notches 506, eight or more notches, or ten or more notches. For instance, the mating interface may include eight notches. In another example, the mating interfaces may include two or more raised portions 312, four or more raised portions 312, or six or more raised portions 312. For instance, the mating interface may include two raised portions 312. In embodiments, the notches 506 are disposed on the male connectors 304, 404 and the raised portions 312, 412 are disposed on the female connectors 302, 402 (e.g., either connector may include notches 506 and raised portions 312).

The straight joint 500a, angulated joint 500b, and hybrid joint 500c may provide different levels of articulation between two adjacent shaft segments 112. For example, for two adjacent shaft segments coupled via a straight joint 500a, the shaft segments 112 may rotate independently of each other, but along the same longitudinal axis (e.g., cylindrical axis). In another example, for two adjacent shaft segments 112 coupled via a hybrid joint 500c, the shaft segments 112 may rotate independently of each other, but along different longitudinal axes at a constant angle (e.g., constant angular displacement) relative to the axes of the connectors. For instance, the hybrid joint 500c may provide an angular displacement of 5° or more, of 10° or more, or 15° or more, of 20° or more, or 25° or more, of 30° or more, of 35° or more, or 40° or more, or of 45° or more.

In another example, for two adjacent shaft segments 112 coupled via an angulated joint 500b, the shaft segments 112 may be rotatable independently of each other along different longitudinal axes and are capable of attaining different levels of angular displacement based on the rotation. For example, a joint 500b having both an angulated female connector 402 and an angulated male connector 404 may, when rotated relative to each other, cause the angle between the coupled shaft segments 112 to change within a range of angles, with the notches 506 securing the joint 500b at specific angles within the range of angles. The number of possible specific angles may depend on the number of notches 506.

The range of angles produced by a rotation of the angulated female connector 402 and a male angulated connector 404 may include or be within a range of 0° to 90°, within a range of 0° to 75°, within a range of 0° to 60°, within a range of 0° and 45°, within a range of 0° and 30°, within a range of 0° and 20°, or within a range of 0° and 10°. For example, the range of angles produced by a rotation of the angulated female connector 402 and a male angulated connector 404 may be or may include a range of 0° to 20°. For instance, for an angulated joint 500b producing a rotation of the angulated female connector 402 and a male angulated connector 404 in a range of 0° to 20°, and having a mating interface that may include eight fixable positions (e.g., based on the number of notches 506), the angulated joint 500b may be configured to cause two adjacently coupled shaft segments 112 to change their angular displacement relative to each other at a set of angles that include one or more of 0° displacement (e.g., straight), 5° displacement, 10° displacement, 15° displacement, 20° displacement, 25° displacement, 30° displacement, 35° displacement, and 40° displacement, or approximate values thereof.

It is noted that the number of different usable configurations of the tool shaft 104 is based on the number or type of joint used. For example, for the tool shaft 104 of FIG. 2A that includes two straight joints 114a, 114c, and two angulated joints 114b, 114d, with each joint 114a-d having eight different stop positions, the tool shaft 104, may have as many as 1600 different lockable configurations.

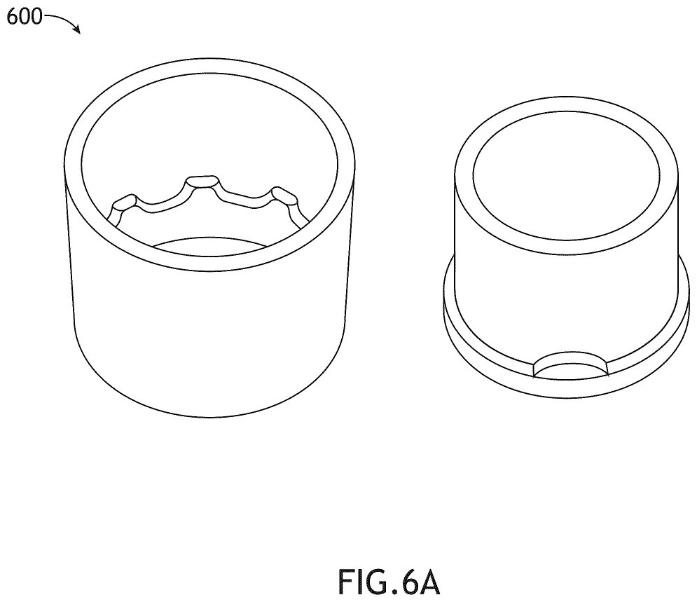

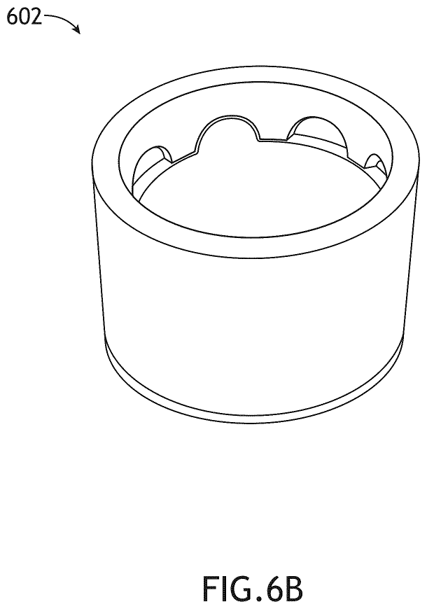

In embodiments, the joints 500 may include other types of interlocking mechanisms for coupling the male and female connectors. For example, a joint 600 may include an expansion lock mechanism where the female and male connectors are unlocked through compression (e.g., pull locking) as shown in FIG. 6A. In another example, a joint 602 may include a compression lock wherein the female and male connectors are unlocked through releasing compression (e.g., push locking), as shown in FIG. 6B. Both pull locking and push locking mechanisms may require one or more springs.

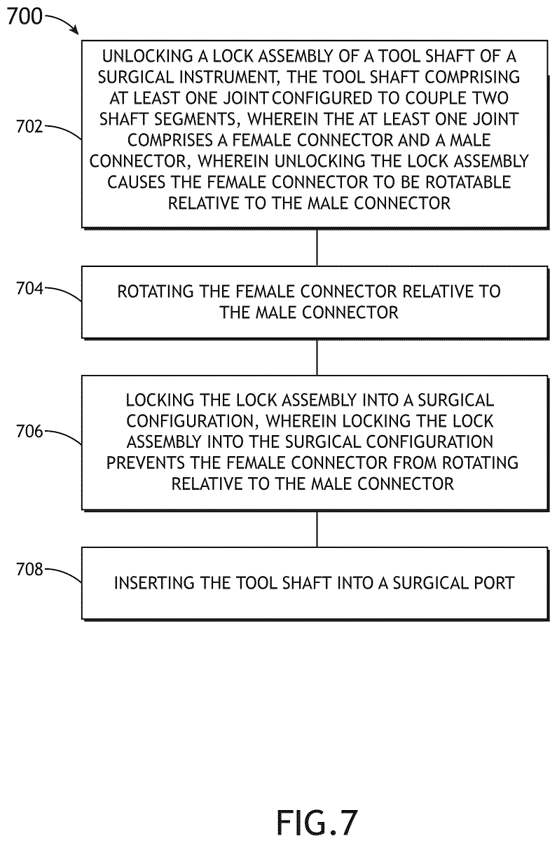

FIG. 7 illustrates a process flow diagram depicting a method 700 for configuring a surgical instrument 100 in preparation for a laparoscopic surgery. The method 700 may be performed using the surgical instrument as described herein.

In embodiments, the method 700 includes a step 702 of unlocking a lock assembly 116 of a tool shaft 104 of the surgical instrument 100, the tool shaft 104 comprising at least one joint 114, wherein the at least one joint comprises a female connector 302 and a male connector, 304 wherein unlocking the lock assembly 116 causes the female connector 302 to be rotatable relative to the male connector 304. The joint 114 may be a straight joint, an angulated joint, or a hybrid joint.

In embodiments, the method 700 includes a step 704 of rotating the female connector relative to the male connector, wherein rotating the female connector relative to the male connector causes the two shaft segments to reconfigure into a surgical configuration. In embodiments, the method 700 includes a step 706 of locking the lock assembly 116 into a surgical configuration, wherein locking the lock assembly 116 into the surgical configuration prevents the female connector 302 from rotating relative to the male connector about the 302. In embodiments, the method 700 includes a step 706 of inserting the tool shaft 104 into a surgical port, such as a surgical port for a laparoscopic surgery.

### EXAMPLES

The following examples are illustrative and should not be interpreted to limit the scope of the claimed subject matter.

Example 1: A modular articulated instrument for manual laparoscopic manipulation.

We envisioned a tool that could achieve custom and variable geometries all along the shaft body. Our goal was to create a manual laparoscopic surgical tool that could achieve triangulation inside the patient, as well as greater tool handle spacing outside the patient, while being compatible with the SILS method. Our approach attempts to bridge between continuum tools and traditional manual straight tools. Our goal is to create an instrument with the capability of achieving the complex geometries of the continuum tool while maintaining the simplicity of the straight manual surgical tool. We pursued a design integrating a series of statically articulating joints along the tool body, which allows a surgeon to create custom tool geometries to suit the needs of the surgery and tool workspace.

The specific landscape of laparoscopic surgery guided how the passively articulating joints could be designed. Surgical tools need to fit through the designated trocar size, and in the single-port method, multiple tools need to fit through the same trocar/port system. This constrains the diameter of the tool as well as its joints. Additionally, the outer body of the tool may need to be smooth, and joints may need to maintain this smooth profile in all phases of bending. Joints may also need to passively maintain rigidity and have a mechanism that allows rotation to be locked throughout the procedure, while retaining ease of use during preoperative adjustment of the joint angles. The result of these constraints is a concentric joint design that allows different bending angles to be achieved through discrete rotational movements. The primary design concept for these joints was an interlocking semicircular notch design, as shown in FIGS. 3A-6B, in which one side of the joint has semicircular extrusions around the outer rim of its cylindrical body, which fit snugly into the semicircular notches on the outer rim of the other side of the joint. The joints articulate through rotational motion. Each side of the joint attaches to its tool shaft segment at a 10-degree angle, for a total potential bending angle of 20 degrees. There are 8 notches within the joint, which allow for 5 positional increments between 0 and 180 degrees of rotation, corresponding to a bending range of 0 to 20 degrees per joint in increments of 5 degrees.

Two different versions of the semicircular notch joint were devised: one in which the two sides of the joint were unlocked through compression (e.g., pull locking, as shown in FIG. 6A), and another in which they were unlocked through releasing compression (e.g., push locking, as shown in FIG. 6A). The pull locking mechanism involved the two sides of the joint being interlocked, with a wave spring around the perimeter of each joint to lock rotation, and in the absence of the tension from the spring, the joint would be able to rotate freely. The use of multiple springs as well as interlocked joints created higher manufacturing complexity. As such, the push lock design was developed as an alternative (see FIGS. 3A-4B). The push lock mechanism reversed the direction of the semicircular protrusions, allowing the joints to fit together without interlocking. As such, different segments can be interchanged modularly along the tool body for a more customizable tool. As this variation was unlocked by being pulled apart, the joint is locked in a fully flush position, which maintains a smooth outer profile during surgery. Rotation is locked by compressing the entire shaft of the tool, and unlocked by releasing this compression.

The rotate-to-bend system of joints allows for a streamlined tool, but creates a set direction for each increment of bend. To address this, a system of alternating joints was devised. Using the same semicircular notch concept, rotational joints were attached without an angled incline to the shaft segments. Placing a straight joint in series before a bending joint allows independent adjustment of the relative orientations of sequential joints in the tool shaft.

The small size of the inner lumen of the device, along with the variable bending and rotation of the shaft segments, necessitated cable actuation for control of both the joints and the grasper tip (or “end effector”). Two separate cables were used, both of which route through the entire shaft and into the handle. The joint cable is responsible for compressing the joints when tensioned, and releasing the joints when relaxed by the surgeon, as shown in FIGS. 1A-1B. The end effector cable runs through the shaft body to the base of the end effector arms. The cable may connect to an antagonist spring, which counteracts the cable to allow for the tool to open and close. The base of each end effector arm was designed to allow a single cable to pass through each to control its back-and-forth motion. These cables then joined with the cable routed through the tool body on one side and the spring on the other.

Laparoscopic tools traditionally use push-rod actuation for their end effectors. The grasper of this tool was actuated by a different method, which accommodates geometry changes while also fitting within the profile of the tool shaft. For simplicity, an actuating cable and a spring antagonist were used to control the opening and closing of the jaws, with the spring returning the jaws to their original position when the cable is released. The spring may either hold the jaws closed with the cable controlling the opening of the jaws, or hold the jaws open at rest with the cable controlling the closing of the jaws.

A prototype was constructed using an aluminum tube and 3D-printed joints, as shown in FIG. 2B. The shaft segment lengths were randomly selected to produce arbitrary overall shaft shapes when deflected. The joint types were alternated (straight, angulated, straight, angulated), allowing local roll orientation of each segment independent of its articulation angle. The end effector itself is on a straight joint base, so it is able to swivel, and it has a bending joint/straight joint pair with a very short segment length to allow for tight bending to emulate the bending and swivel movement of other laparoscopic tools.

To unlock the joints for tool geometry manipulation, cable tension is released via a lever on the top of the handle, as shown in FIGS. 1A-1B. where it can be controlled by the surgeon's thumb. As the bending joints throughout the tool body are manipulated, the length of the cable path changes, altering tension through the cable. As such, there was a need to have increments of locking rather than a binary locked and unlocked position. This was achieved through a ratchet locking system (e.g., lock assembly 116) with seven increments (e.g., ratchet positions 146). This system also allows for a small amount of tension to be released in the cable such that only one joint is able to separate enough to rotate at a time. This makes the manipulation of the joints to set tool geometries easier, as the rest of the tool body will stay rigid and stable. Both the locking of the joints and the control of the end effector rely on appropriate cable tension, which means that both cables need to be tensioned and relaxed together to achieve joint movement and end actuation. To achieve this, both cables are routed around pulleys (e.g., pulley wheel 138) that are attached to the locking lever (e.g., pulley lever 132). The joint locking cable is then anchored within the handle, while the end effector actuator cable routes to the moving handle. The handle can then be used to apply additional tension to this cable alone to close the jaws of the end effector.

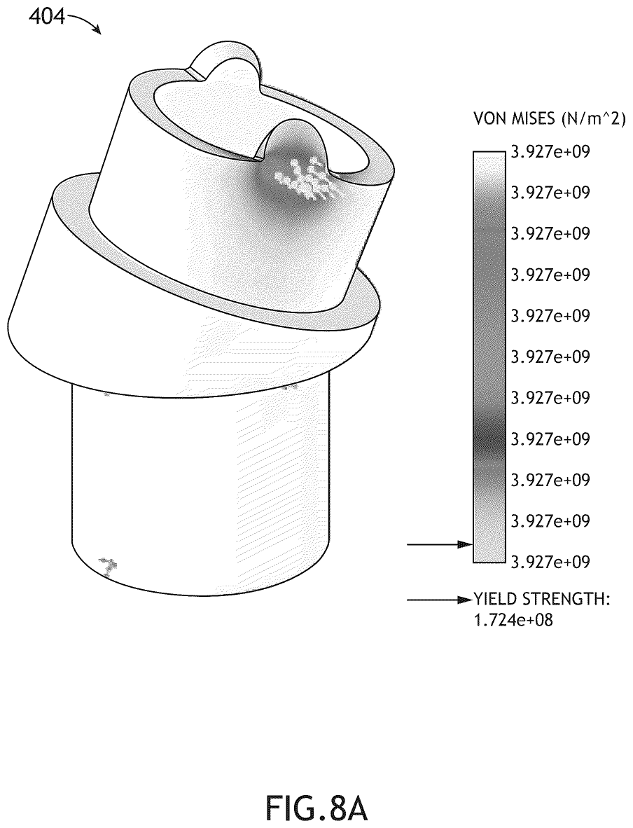

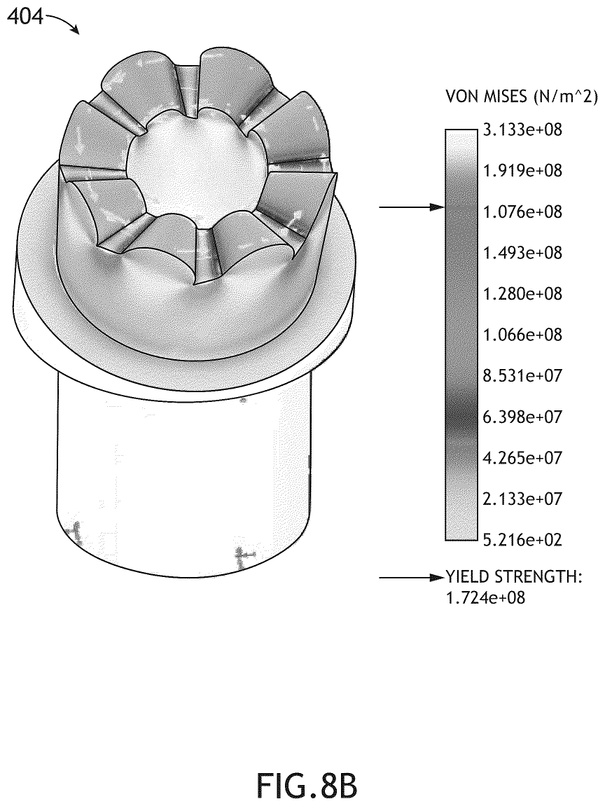

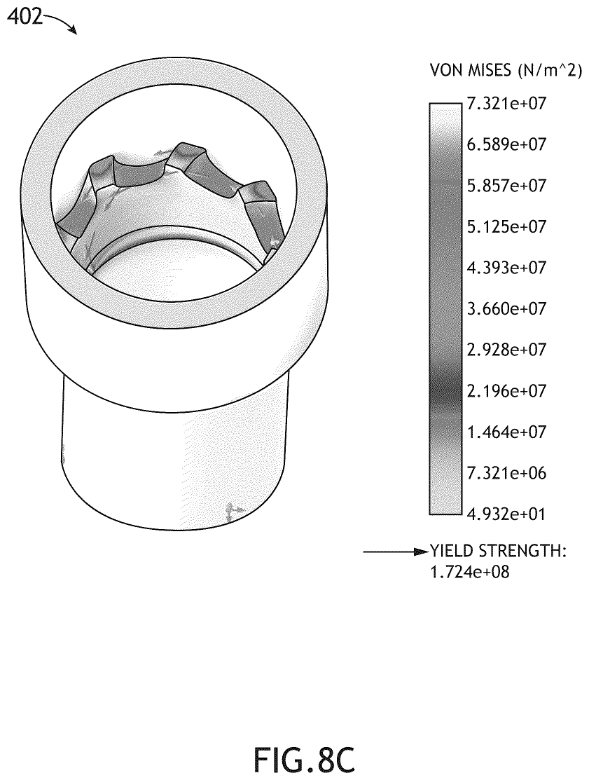

The strength of the device was validated through the use of the SolidWorks® (Dassault Systèmes) finite element analysis (FEA) feature, as shown in FIGS. 8A-8C. Each joint type was tested under a simulated torsional load of 2 Nm, which correlates to a typical safe load for patient tissues during surgery applied at an offset distance commensurate with the device's angulated joints. The simulated joints were made of 316 stainless steel. The original design of the joints included two semicircular lobes on opposite sides of the perimeter of the male half, with an open lumen for cable routing. This design showed high levels of stress under this loading, such that the design was reconfigured to have a 1:1 ratio of semicircular lobes and notches, and the lumen size was reduced on both sides of the joint to allow for a thicker wall. These changes resulted in a joint with reduced stress concentration that would be able to theoretically withstand maximum loading during surgery.

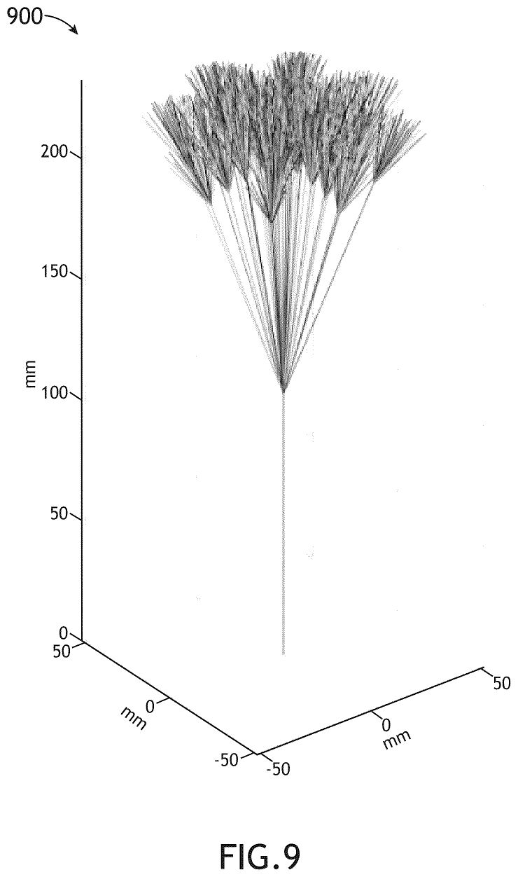

The achievable configurations were also quantified in simulation using MATLAB® (Mathworks, Inc.) based on a tool with two straight and two bending joints (e.g., similar to how the physical prototype was implemented). This demonstrated a total of 1600 different configurations, as shown in FIG. 9.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

Although particular embodiments of this invention have been illustrated, it is apparent that various modifications and embodiments of the invention may be made by those skilled in the art without departing from the scope and spirit of the foregoing disclosure. It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. Accordingly, the scope of the invention should be limited only by the claims appended hereto.

What is claimed:

1. A surgical instrument comprising:
a tool handle comprising a tool control;
a tool shaft coupled at a proximal end to the tool handle, the tool shaft comprising:
a plurality of shaft segments, wherein the plurality of shaft segments comprise a lumen extending along a length of the plurality of shaft segments; and
a plurality of joints configured to couple adjacent shaft segments of the plurality of shaft segments, one or more joints of the plurality of joints comprising:
a male connector comprising a male mating surface; and
a female connector comprising a female mating surface and configured to couple to the male connector at a mating interface, wherein a rotation of the male connector relative to the female connector causes an articulation of a tool relative to the tool handle;
a tool disposed on distal end of the tool shaft and mechanically coupled to the tool control, wherein actuation of the tool control causes the tool to perform a function; and
a lock assembly disposed on the tool handle and configured to lock the tool shaft, wherein locking the tool shaft prevents the male connector from rotating relative to the female connector.

2. The surgical instrument of claim 1, the lock assembly comprising:
a pulley lever disposed on the tool handle;
a pulley wheel mechanically coupled to the pulley wheel, and
a joint cable coupled to the tool handle at a first anchor point and to the distal end of the tool shaft via the lumen, the joint cable configured to compress the plurality of shaft segments when the pulley lever is actuated, wherein a compression of the plurality of shaft segments prevents the male connector from rotating relative to the female connector.

3. The surgical instrument of claim 1, wherein one joint of the plurality of joints comprises a straight joint, wherein the rotation of the male connector relative to the female connector causes a shaft segment coupled to the male connector and another shaft segment coupled to the female connector to rotate relative to each other along a shared longitudinal axis.

4. The surgical instrument of claim 1, wherein one joint of the plurality of joints comprises an angulated joint, wherein the rotation of the male connector relative to the female connector causes a shaft segment coupled to the male connector and another shaft segment coupled to the female connector to rotate relative to each other along different longitudinal axes.

5. The surgical instrument of claim 4, wherein a rotation of the male connector relative to the female connector causes the shaft segment and the another shaft segment to switch between a plurality of angled configurations from a straight configuration to 20 degrees from the straight configuration.

6. The surgical instrument of claim 5, wherein another one joint of the plurality of joints comprises a straight joint, wherein the rotation of the male connector of the straight joint relative to the female connector of the straight joint causes a shaft segment coupled to the male connector of the straight joint and another shaft segment coupled to the female connector of the straight joint to rotate relative to each other along a shared longitudinal axis.

7. The surgical instrument of claim 1, wherein the tool shaft comprises at least five shaft segments.

8. The surgical instrument of claim 1, wherein at least one of the male connector or female connector further comprises:
a support section configured to fit within the lumen of a first shaft segment of the plurality of shaft segments; and
a base section configured to support a mating surface of the mating interface.

9. The surgical instrument of claim 1, wherein the mating interface of a joint of the plurality of joints comprises:
a plurality of notches disposed on one of the male connector or the female connector; and
at least one raised portion disposed on an other of the male connector and the female connector, wherein the plurality of notches and the at least one raised portion are configured to engage with each other.

10. The surgical instrument of claim 9, wherein one or more notches of the plurality of notches comprise semicircular notches.

11. The surgical instrument of claim 1, wherein the tool control is mechanically coupled to the tool via a control cable.

12. The surgical instrument of claim 11, further comprising:
a pulley lever disposed on the tool handle;
a pulley wheel mechanically coupled to the pulley wheel, and
a joint cable coupled to the pulley lever at the distal end of the tool shaft via the lumen, the joint cable configured to compress the plurality of shaft segments when the pulley lever is actuated, wherein a compression of the plurality of shaft segments prevents the male connector from rotating relative to the female connector, wherein an actuation of the pulley lever causes an adjustment of a tension of the control cable.

13. The surgical instrument of claim 11, wherein the tool comprises a grasper, wherein a pulling of the control cable causes one or more jaws of the grasper to close.

14. The surgical instrument of claim 12, wherein the tool comprises scissors.

15. The surgical instrument of claim 11, wherein the tool comprises a clamp.

16. The surgical instrument of claim 11, wherein the tool control comprises a trigger mechanically coupled to the control cable.

17. The surgical instrument of claim 1, wherein the surgical instrument is configured for insertion into a laparoscopic port.

18. The surgical instrument of claim 1, wherein the plurality of shaft segments comprises:
a proximal shaft segment coupled to the tool handle; and
a distal shaft segment coupled to the tool, wherein upon an adjustment of the plurality of joints, the proximal shaft segment and the distal shaft segment are configured to be angularly displaced in a range of zero degrees to 60 degrees.

19. The surgical instrument of claim 1, wherein the tool shaft comprises at least five shaft segments.

20. A method for configuring a surgical instrument in preparation for a laparoscopic surgery comprising:
unlocking a lock assembly of a tool shaft of the surgical instrument, the tool shaft comprising at least one joint configured to couple two shaft segments, wherein the at least one joint comprises a female connector and a male connector, wherein unlocking the lock assembly causes the female connector to be rotatable relative to the male connector;
rotating the female connector relative to the male connector, wherein rotating the female connector relative to the male connector causes the two shaft segments to reconfigure into a surgical configuration. locking the lock assembly into the surgical configuration, wherein locking the lock assembly into the surgical configuration prevents the female connector from rotating relative to the male connector; and
inserting the tool shaft into a surgical port.
